(19) United States
(12) Patent Application Publication
(10) Pub. No.: US 2006/0014801 A1
(43) Pub. Date: Jan. 19, 2006

(54) Prevention and treatment of cognitive impairment using (R)-(-)-5-methyl-1-nicotynoyl-2-pyrazoline (MNP) and analogs

(75) Inventors: Michela Gallagher, Baltimore, MD (US); Pauline Kay Lund, Carrboro, NC (US); Joel C. Selcher, San Mateo, CA (US); Thorsten Melcher, San Francisco, CA (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Johns Hopkins University, Baltimore, MD (US)

(21) Appl. No.: 11/058,734

(22) Filed: Feb. 14, 2005

Related U.S. Application Data
(63) Continuation-in-part of application No. 10/722,357, filed on Nov. 24, 2003.
(60) Provisional application No. 60/428,229, filed on Nov. 22, 2002.

(51) Int. Cl. A61K 31/4439; C07D 403/02

(52) U.S. Cl. 514/341; 546/272.1; 546/275.4

(57) ABSTRACT

The invention provides methods for improving cognitive function in a subject by administering (R)-(−)-5-methyl-1-nicotinoyl-2-pyrazoline (MNP) or an analog to a subject in need of such treatment. The invention is useful for treatment of cognitive impairment such as mild cognitive impairment (MCI) as well as other conditions.

### GOVERNMENT SUPPORT

This invention was made with government support under grant No. PO1 AG09973 awarded by the National Institutes on Aging. The government may have certain rights in the invention.

### BACKGROUND OF THE INVENTION

Some decline in cognitive ability may be a normal consequence of aging. However, a significant population of elderly adults experiences a decline in cognitive ability that exceeds normal aging. Many of those individuals are diagnosed as suffering from Alzheimer's Disease (AD), which is estimated to afflict four million individuals in the United States. Others exhibit cognitive decline that is of insufficient magnitude to warrant a diagnosis of AD, but may be diagnosed as suffering from Age-Related Cognitive Decline (ARCD) or Mild-Cognitive Impairment (MCI). There are many other conditions (including Huntington's Disease, Parkinson's Disease, Multiple Sclerosis, schizophrenia, depression, Lewy body dementia, vascular dementia, HIV associated dementia and other types of dementias) in which cognitive impairment is a component and contributes to the disability of the afflicted individuals.

Although a limited number of drugs are now available that may improve cognition in Alzheimer's Disease, there is a great need for additional drug treatments and therapeutic approaches for improving cognition in patients with AD. In addition, there is an urgent need for new treatments to improve cognitive function in patients diagnosed with MCI, ARCD and similar age-associated impairments. This invention meets these and other needs.

### SUMMARY OF THE INVENTION

In one aspect the invention provides a method for improving cognitive function in a subject, comprising administering a therapeutically effective amount of (R)-(−)-5-methyl-1-nicotinoyl-2-pyrazoline (MNP; also called MS-153), or an analog or derivative thereof, to a subject in need of such improvement. In one embodiment, the subject exhibits age-related cognitive decline. In one embodiment, the subject is a human patient diagnosed with, or suspected of having, cognitive impairment due to Alzheimer's Disease (AD), Mild Cognitive Impairment (MCI) or Age Related Cognitive Decline (ARCD). In an embodiment, the subject is not diagnosed with or under treatment for stroke.

In an aspect, MNP, analog or derivative is administered in combination with another agent effective for treatment for cognitive impairment. The other agent may be, for example, a GABA_{B} receptor antagonist, an acetylcholinesterase inhibitor, or an NMDA receptor antagonist. In one embodiment, the MNP, analog or derivative and the other agent are administered at the same time or as a co-formulation.

In one embodiment MNP is administered orally at a daily dosage of from 100 mg/day to 700 mg/day to a patient diagnosed with, or suspected of having, cognitive impairment due to Alzheimer's Disease (AD), Mild Cognitive Impairment (MCI) or Age Related Cognitive Decline (ARCD). In an embodiment, the MNP is administered for at least two months.

### BRIEF DESCRIPTION OF THE DRAWINGS

FIGS. 1A-1C show the effect on GLT1 protein expression in the hippocampus of young rats of subcutaneous administration of MNP for 7 days. FIG. 1A shows Western blots of hippocampal tissue from rats treated with vehicle (saline) or 50 mg/kg/day MNP (MNP-50) for 7 days. GLT1 immunoreactivity was significantly higher in the MNP-treated animals compared to vehicle controls. There was no difference between the two groups in the level of GLT1B immunoreactivity. Coomassie blue staining (Stain) showed equal protein loading across all samples. FIGS. 1B and 1C are summary histograms illustrating the significant increase in GLT1 (FIG. 1B) but not GLT1B (FIG. 1C) protein expression induced by 7 days of treatment with 50 mg/kg/day MNP.

FIGS. 2A-B show the improvement in spatial memory retention in aged impaired (AI) rats treated with MNP. FIG. 2A plots the swim path length (in cm) required to locate an escape platform for vehicle- and MNP-treated rats over the course of six training trials and a retention trial. FIG. 2B shows the mean savings score, a measure of memory retention, for vehicle- and MNP-treated groups of aged impaired Long-Evans rats tested in the spatial working memory version of the Morris water maze.

### DETAILED DESCRIPTION OF THE INVENTION

### I. Definitions

For the convenience of the reader, certain terms employed in the specification, examples, and appended claims are collected here. Unless defined otherwise, all technical and scientific terms used herein have the same meaning as commonly understood by one of ordinary skill in the art to which this invention belongs.

“Cognitive function” refers to higher order intellectual, brain processes involved in learning and memory, including, but not limited to, attention, short-term memory, long-term memory and memory acquisition, consolidation and retrieval, and expressing an interest in surroundings and self-care. In humans, cognitive function may be measured, for example and without limitation, by the Alzheimer's Disease Assessment Scale-cognitive subscale (ADAS-cog); the clinical global impression of change scale (CIBIC-plus scale); the Alzheimer's Disease Cooperative Study Activities of Daily Living Scale (ADCS-ADL); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). See Folstein et al., 1975, The “mini-mental state”: a practical method for grading the cognitive state of patients for the clinician. J Psychiatric Res 12: 189-98; Robbins et al., 1994, Cambridge Neuropsychological Test Automated Battery (CANTAB): A factor analytic study of a large sample of normal elderly volunteers. Dementia 5: 266-81; Rey, 1964, L'examen clinique en psychologie. Paris: Presses Universitaires de France; Wechsler, 1987, Wechsler Memory-Scale-Revised. New York: Psychological Corporation; Kluger et al., 1999, Neuropsychological prediction of decline to dementia in nondemented elderly. J Geriatr Psychiatry Neurol 12:168-79. In animal model systems, cognitive function may be measured any number of ways known in the art, including using the following apparati: Morris water maze, Barnes circular maze, elevated radial arm maze, T maze or any other mazes in which subjects use spatial information. Other tests known in the art may be used to assess cognitive function, such as fear conditioning, novel object recognition, active avoidance, illuminated open-field, dark activity meter, elevated plus-maze, two-compartment exploratory test or forced swimming test. In addition, cognitive function may be measured using imaging techniques such as Positron Emission Tomography (PET), functional magnetic resonance imaging (fMRI), Single Photon Emission Computed Tomography (SPECT), or any other imaging technique that allows one to measure brain function.

“Impaired cognitive function” refers to cognitive function that is not as robust as that observed in an age-matched normal subject and includes states in which cognitive function is reduced. In some cases, cognitive function is reduced by about 5%, about 10%, about 30%, or more, compared to cognitive function measured in an age-matched normal subject. Impaired cognitive function may be associated with many diseases or disorders, involving dementias (e.g. Lewy body dementia, vascular dementia, Alzheimer's Disease, and HIV associated dementia), Huntington's Disease, Parkinson's Disease, Multiple Sclerosis, schizophrenia, depression, Mild Cognitive Impairment (MCI), Age-Associated Memory Impairment (AAMI), and Age Related Cognitive Decline (ARCD).

“Promoting” cognitive function refers to affecting impaired cognitive function so that it more closely resembles the function of an aged-matched normal, unimpaired subject, and includes affecting states in which cognitive function is reduced compared to a normal subject. Cognitive function may be promoted to any detectable degree, but in humans preferably is promoted sufficiently to allow an impaired subject to carry out daily activities of normal life.

“Preserving” cognitive function refers to affecting normal or impaired cognitive function such that it does not decline or does not fall below that observed in the subject upon first presentation or diagnosis, e.g., to the extent of expected decline in the absence of treatment.

“Improving” cognitive function means promoting cognitive function and/or improving cognitive function in a subject.

“Subject” refers to a mammal. In an embodiment, the subject is a human. In another embodiment, the subject is a rat. In another embodiment, the subject is an experimental model animal such as a non-human primate, ovine, bovine, porcine, equine, feline, murine or canine.

“Treating” impaired cognitive function in a subject or “treating” a subject having impaired cognitive function are used herein to refer to providing the subject with a therapeutic agent by any appropriate means, e.g., the administration of a drug, such that at least one symptom of the impaired cognitive function is stabilized or decreased. Treating impaired cognitive function can comprise preventing the impairment, delaying progression of the impairment, slowing the rate of deterioration in cognitive function or improving the impairment (lessening disease severity) or curing the impairment. In the context of impaired cognitive function the presence or degree of therapeutic effect can be assessed using standard behavioral or other tests known in the art for assessing cognitive function.

### II. (R)-(−)-5-methyl-1-nicotinoyl-2-pyrazoline (MNP, MS-153)

In an aspect, the invention relates to a method for improving cognitive function in a subject in need of such improvement, comprising administering a therapeutically effective amount of (R)-(−)-5-methyl-1-nicotinoyl-2-pyrazoline (MNP) or an analog thereof to the subject.

MNP has been investigated as a potential cerebral anti-ischemic agent. MNP is described in U.S. Pat. No. 5,089,622, which is incorporated herein by reference. Also see Shimada et al., 1999, Eur J Pharmacol. 386:263-70; Kawazura et al., 1991, “Effects of MS-424, a novel anti-cerebral ischemic agent, on focal cerebral ischemia in rats”, Jpn J Pharmacol, 55 (Suppl. I): Abst P-428; Takahashi et al., 1991, “Anti-cerebral anoxic and anti-global cerebral ischemic actions of a novel pyrazoline derivative, MS-424”, Jpn J Pharmacol, 55 (Suppl. I): Abst P-427; Nakamura et al., 1992, “The effect of a novel anti-cerebral ischemic agent, MNP, on cerebral infarction in dogs”, Jpn J Pharmacol, 58(Suppl. 1): Abst P-025; Kawazura et al., 1993, “Anti-ischemic effects of MNP: Effects of neurological deficits and cerebral infarction in MCA occluded rats”, J Cereb Blood Flow Metab, 13(Suppl. 1): S699; Akaike et al., 1993, “Protection by pyrazoline analog MNP against glutamate cytotoxicity in cultured cortical neurons”, J Cereb Blood Flow Metab, 13 (Suppl. 1): S663; “Mitsui Toatsu and Mitsui Chemical's started clinical trials with a new protecting agent for cerebral hemorrhage”, Nikkan Kogyo Shinbun Jun. 23, 1993: Umemura et al., 1994, “Effect of the anti-ischaemic agent, MNP, alone and in combination with the tissue-type plasminogen activator on the ischaemic rat cerebral lesions”, Jpn J Pharmacol, 64 (Suppl. 1): Abst O-199; Sekikawa et al., 1995 “Effects of MNP, a new neuroprotective agent, on the recovery of spinal reflex potentials after spinal cord ischemia in cats”, Jpn J Pharmacol, 67 (Suppl. 1): Abst P1-279; Kosuge et al., 1995, “Effect of MNP, in ischaemic lesions in the rat middle cerebral artery occlusion model”, Jpn J Pharmacol, 67 (Suppl. 1): Abst P3-121; “Mitsui's new treatment agent for acute phase of cerebral infarction enter late phase II stage”, Kagaku Kogyo Nippo 1996: February 23; Mitsui Pharmaceuticals Inc. Annual Report; Umemura et al., 1996, “Inhibitory effect of MNP on elevated brain glutamate level induced by rat middle cerebral artery occlusion”, Stroke 27(9): 1624; Shiga et al., 1996, “Cerebroprotective effect of MNP after reperfusion or permanent focal ischemia in the rat”, Jpn J Pharmacol, 73(Suppl. 1): Abst P-511; Shimada et al., 1997, “Inhibitory effect of MNP, a cerebroprotective agent, on glutamate release from the rat hippocampal slice”, Jpn J Pharmacol, 73 (Suppl. 1): Abst P-512; Sakata et al., 1997, “Effect of MNP, a cerebroprotective agent, on gamma protein kinase C redistribution in rat hippocampal slices”, Jpn J Pharmacol, 73 (Suppl. 1): Abst P-513; Kawazura et al., 1997, “Cerebroprotective effects of a novel pyrazoline derivative, MNP, on focal ischemia in rats”, Jpn J Pharmaco, 73 (4): 317; Shimada et al., 1997, “Inhibitory effect of MNP, a neuroprotective agent, on glutamate efflux through glutamate transporter”, Soc Neurosci Abst, 24 (Part 1) Abst 382.4; Shimada et al., 1998, “The neuroprotective agent MNP stimulates glutamate uptake”, Eur J Pharmacol, 386 (2-3): 263; Honda et al., 1999, “Effect of a novel pyrazoline derivative, MNP on methamphetamine or cocaine-induced locomotor activity of rats”, Soc Neurosci Abst 2000, 26(Part 1) Abst 473.13.

### III. Subjects

In an aspect of the invention, MNP or an analog or derivative is administered to a subject in need of improvement of cognitive function. In one aspect, the subject has impaired cognitive function. In one aspect, the subject has impaired cognitive function due to a condition associated with aging (such as, Mild Cognitive Impairment, Age Related Cognitive Decline; or Alzheimer's Disease). Signs and symptoms of these disorders are well known, and it is within the skill of medical professionals to diagnose such disorders with reference to the medical literature, and thereby identify individuals with a disorder. Diagnosis may be aided by reference to (1) DIAGNOSTIC AND STATISTICAL MANUAL OF MENTAL DISORDERS (4th Edition, American Psychiatric Association (hereinafter “DSM-IV”) incorporated by reference herein; (2) The International Statistical Classification of Diseases and Related Health Problems, Tenth Revision (hereinafter “IDC-10”) incorporated by reference herein; (3) the medical literature.

For example, a diagnosis of ARCD (or the equivalent construct such as age-associated Memory Impairment) is used to define patients with a mild memory deficit that is not expected to worsen considerably over time. ARCD can also be defined as Stage 2 on the Global Deterioration Scale (GDS). The GDS is a seven-point rating system of cognitive and functional capabilities, widely used for rating cognitive performance in older adults, with scores ranging from normal aging (Stage 1) to severe dementia (Stage 7). Stage 2 is characterized by the following clinical characteristics: subjective cognitive complaints in the absence of clinically manifest deficit.

Mild Cognitive Impairment (MCI) is a condition characterized by isolated memory impairment accompanied by no other cognitive abnormality and relatively normal functional abilities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines). See Petersen et al., 1999, Mild cognitive impairment: clinical characterization and outcome. Srch. Neurol. 56: 303-308. Also see Petersen, 2003, Mild cognitive impairment: Aging to Alzheimer's Disease. New York: Oxford University Press.

MCI can also be defined as Stage 3 on the Global Deterioration Scale (GDS). Stage 3 is characterized by the following clinical characteristics: subtle, clinically manifest cognitive impairment that may be of sufficient magnitude to interfere with complex occupational or social tasks which may be accompanied by anxiety.

MCI can also be defined as a rating of 0.5 on another widely used system for rating cognitive and functional capabilities, the Clinical Dementia Rating (CDR) scale. Scores in the CDR scale range from a CDR assignment of 0 (no dementia) to 3 (severe dementia). The degree of impairment in performance is assessed within six categories of cognitive functioning: memory, orientation, judgment/problem solving, community relations, home and hobbies, and personal care. MCI subjects also have significantly greater psychometric test deficits (Reisberg et al., 1982, The global deterioration scale for assessment of primary degenerative dementia. Am J Psychiatry 139:1136-39) balance and coordination deficits (Franssen et al., 1999, J Am Geriatric Soc 47:463-99) and deficits on motor performance tasks (Kluger et al., 1997, J Gerontology: Psychol. Sci. 52B: 28-39) than AAMI and normal aged subjects.

Based on these operational definitions, a diagnosis of MCI requires an objective assessment of cognitive impairment, which can be garnered through the use of well-established neuropsychological tests, including the Mini Mental State Examination (MMSE), the Cambridge Neuropsychological Test Automated Battery (CANTAB) and individual tests such as Rey Auditory Verbal Learning Test (AVLT), Logical Memory Subtest of the revised Wechsler Memory Scale (WMS-R) and the New York University (NYU) Paragraph Recall Test.

In a related aspect of the invention, the subject has impaired cognitive function associated with Huntington's Disease, Parkinson's Disease, Multiple Sclerosis, schizophrenia, depression, Lewy body dementia, vascular dementia, HIV associated dementia and other types of dementias. The aforementioned conditions are known in the medical art and can be recognized by a physician of ordinary skill. In these conditions, the degree of impairment may increase with age.

In another aspect of the invention, the subject has impaired cognitive function associated with emotional stress or acute brain trauma. In another aspect of the invention the subject does not have impaired cognitive function, but desires enhanced cognitive function.

According to the present invention, it is generally the case that the subject is not under treatment for stroke. Thus, generally the subject is not diagnosed as having had a stroke within the last year, or under the care of a physician for a stroke, its sequelae or effects. As used herein, “stroke” has its usual meaning in the medical art, i.e., a lesion resulting from a cerebral ischemic or hemorrhagic event. In a related embodiment, the subject is not under treatment for or diagnosed as having a cerebrovascular accident (CVA) for example, cerebral insufficiency, cerebral infarction, hemorrhage, or arteriovenous malformation. In a related embodiment, the subject is not under treatment for cerebrovascular accident (CVA) for example, one or more of cerebral insufficiency, cerebral infarction, hemorrhage, or arteriovenous malformation. See the Merck Manual of Diagnostics and Therapy 17th Edition (1992) Merck and Co. New Jersey, incorporated herein by reference.

### IV. Administration of MNP or Analogs

In an aspect of the invention, MNP or an analog or derivative is administered to a subject in need of improvement of cognitive function. This section describes, for illustration and not limitation, various forms, routes and dosages that may be used.

A. Route of Administration

Pharmaceutical compositions containing MNP, analogs or derivatives may be administered by any number of routes including, but not limited to, oral, parenteral (e.g., intravenous, intramuscular, intra-arterial, intramedullary, intrathecal, intraventricular, subcutaneous, intraperitoneal, intraspinal), transdermal, subcutaneous, intraperitoneal, intranasal, enteral, topical, sublingual, or rectal means.

In reports related to investigation of MNP for treatment of stroke, the compound was administered parenterally (intravenously). Although MNP may be parenterally administered to improve cognitive function, in a preferred embodiment, MNP is administered by a different route (e.g., orally).

B. Pharmaceutical Compositions

MNP, analog or derivative may be administered alone as a pharmaceutical composition to improve cognition, but more usually is administered as a pharmaceutical composition that contains, in addition to the active agent, e.g., MNP, analog or derivative, suitable pharmaceutically-acceptable carriers. The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combining active compounds with solid excipient and, optionally, other compounds. Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. For topical or nasal administration, penetrants appropriate to the particular barrier to be permeated are used in the formulation. Such penetrants are generally known in the art.

After pharmaceutical compositions have been prepared, they can be placed in an appropriate container and labeled for treatment of an indicated condition. For administration of MNP, for example, such labeling could include amount, frequency, and method of administration.

Further details on techniques for formulation and administration may be found in the latest edition of Remington's Pharmaceutical Sciences (Maack Publishing Co., Easton, Pa.); GOODMAN AND GILMAN'S: THE PHARMACOLOGICAL BASIS OF THERAPEUTICS 10^{TH} EDITION 2001 by Louis Sanford Goodman et al., McGraw-Hill Professional; PHARMACEUTICAL DOSAGE FORMS AND DRUG DELIVERY SYSTEMS 7^{th} Edition Howard C. Ansel, et al., 2004, Lippincott Williams & Wilkins Publishers; PHARMACEUTICAL CALCULATIONS 11^{th} Edition, 2001, by Mitchell J. Stoklosa et al., Lippincott Williams & Wilkins; PHYSICAL PHARMACY: PHYSICAL CHEMICAL PRINCIPLES IN THE PHARMACEUTICAL SCIENCES 4^{th} Edition by Pilar Bustamante, et al., 1993, Lea & Febiger.

C. Dosage

According to the invention, a therapeutically effective dose of drug (MNP, analogs or derivatives) is administered to a subject (e.g., human) to improve cognition. “A therapeutically effective dose” refers to an amount of active ingredient sufficient to ameliorate the symptoms or condition. Thus, a therapeutically effective dose of MNP refers to the amount of MNP that will improve, promote or preserve cognitive function in an individual in need of such improvement (e.g., an individual with MCI). It is expected that improvement in cognitive function will result from multiple administrations. Thus, a therapeutically effective dose may be a dose that results in improved cognitive function when administered over an extended period of time (e.g., daily for several months).

Normal dosage amounts may vary from about from 0.1 μg to 10 g, such as from 0.1 μg to 100,000 μg up to a total dose of about 1, 5 or 10 grams, depending upon the route of administration. Guidance as to particular dosages and methods of delivery is provided in the literature and generally available to practitioners in the art.

An animal model may also be used to determine the appropriate concentration range and route of administration. Such information can then be used to determine useful doses and routes for administration in humans. A particularly preferred animal model uses behaviorally characterized rats as described herein. As shown in Example 1, MNP administered subcutaneously to aged rats was effective in improving cognitive ability. The human equivalent dose can be estimated as about 8 mg/kg/day, based on a body-surface-area conversion factor of 6.2. In addition, using other behavioral measures in the rat a lower dose, i.e., 10 mg/kg/day was efficacious. Thus, in one embodiment the parenterally administered dose for a human subject in need of treatment to improve cogntive ability is in the range of about 0.08 mg/kg/day to about 800 mg/kg/day; for example 0.8 mg/kg/day to about 80 mg/kg/day; for example between about 1 mg/kg/day and 50 mg/kg/day. In an embodiment a patient of average weight receives a daily dose of about 5 mg to 1 g, more often 50 mg to 1000 mg, e.g., from about 50 mg to about 600 mg. In an embodiment, the dose is 500 mg/day.

As shown in Example 2, MNP has been demonstrated to be highly bioavailable when administered orally. Exemplary daily doses for human patients can be estimated using the animal pharmacokinetic studies described in Example 2. Based on the results shown, MNP may be as much as about 80% orally bioavailable. Thus, about 125% of the s.c. dose may be efficacious. Thus, in one embodiment the parenterally administered dose for a human subject in need of treatment to improve cognitive ability is in the range of about 1.0 mg/kg/day to about 100 mg/kg/day, for example between about 1.25 mg/kg/day and 65 mg/kg/day. In an embodiment, the daily dose is about 65 mg to 1250 mg, e.g., from about 65 mg to about 750 mg. In one embodiment, the oral dose between 100 mg/day and 700 mg/day for a 60-70 kg patient.

It will be appreciated that the figure above are estimates and the present invention is in no manner limited to the particular dosage ranges described above. The exact dosage will be determined by the practitioner, in light of factors related to the subject requiring treatment. Dosage and administration are adjusted to provide sufficient levels of the active moiety or to maintain the desired effect. Factors which may be taken into account include the degree of cognitive impairment, the general health of the subject, the age, weight, and gender of the subject, time and frequency of administration, drug combination(s), reaction sensitivities, and response to therapy.

Optimal therapeutic doses for MNP analogs or derivatives can be estimated from the doses used for MNP. As noted above, for any compound, the therapeutically effective dose can be estimated initially either in cell culture assays, e.g., according to the method of Aronica et al., supra, or in animal models such as mice, rats, rabbits, dogs, or pigs.

Although daily dosages are described above, it will be understood that, as described below, a variety of administration regimens can be used such as daily; weekly; every other day; 5 days on, 2-days off; or essentially continuous dosing can be used. Thus, MNP, analogs, and derivatives may be administered continuously, daily (in a single or multiple doses), or less often. For example, long-acting pharmaceutical compositions may be administered every 3 to 4 days, every week, or biweekly depending on the half-life and clearance rate of the particular formulation. The drug may be administered as frequently and for as long as needed to have or maintain a therapeutic effect. For example, the drug can be administered for at least two consecutive days, at least three consecutive days, at least five consecutive days or longer, and/or for periods of at least one week, at least two weeks, or at least three weeks. In an embodiment the drug is administered periodically (e.g., daily) for at least four weeks. In some cases the drug will be administered for several months or even years.

### V. MNP Prodrugs, Analogs, Derivatives

Based on the discovery that administration of MNP beneficially affects cognitive ability in impaired subjects it is contemplated that MNP prodrugs, analogs and derivatives can also be administered to improve cognitive function.

The term “prodrug” is art-recognized and is intended to encompass compounds which, under physiological conditions, are converted into MNP or functionally active MNP analog. A common method for making a prodrug is to select moieties which are hydrolyzed under physiological conditions to provide the desired compound. In other embodiments, the prodrug is converted by an enzymatic activity of the host animal.

“Analog” is used herein to refer to a compound which functionally resembles another chemical entity, but does not share the identical chemical structure. For example, an analog is sufficiently similar to base compound that it can substitute for the base compound in therapeutic applications, despite minor structural differences.

“Derivative” is used herein to refer to the chemical modification of a compound. Chemical modifications of a compound can include, for example, replacement of hydrogen by an alkyl, acyl, or amino group. Many other modifications are also possible. A derivative of a compound retains at least one functional property of the original compound.

It will be appreciated that compounds used in the methods of the present invention preferably should readily penetrate the blood-brain barrier when peripherally administered. Compounds which cannot penetrate the blood-brain barrier, however, can still be effectively administered directly into the central nervous system, e.g., by an intraventricular route.

MNP analogs that may be used to improve cognition include compounds of the formula I: 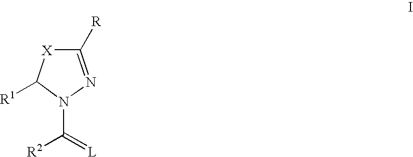
wherein, independently for each occurrence:
R is H, C₁-C₁₀ alkyl, C₂-C₁₀ alkenyl, C₂-C₁₀ alkynyl, aryl, or aralkyl;
R¹ is H, C₁-C₁₀ alkyl, C₂-C₁₀ alkenyl, C₂-C₁₀ alkynyl, aryl, or aralkyl;
R² is a heterocyclic or heteroaryl ring comprising from 1-4 heteroatoms selected from the following: N, O, or S;
L is O, S, or NR; and
X is CR₂, O, or S.

Also included in the methods of the present invention are pharmaceutically acceptable addition salts and complexes of the compounds of formula I. In cases wherein the compounds may have one or more chiral centers, unless specified, the present invention comprises each unique racemic compound, as well as each unique nonracemic compound.

In cases in which the compounds have unsaturated carbon-carbon double bonds, both the cis (Z) and trans (E) isomers are within the scope of this invention. In cases wherein inhibitors may exist in tautomeric forms, such as keto-enol tautomers, such as 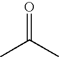
and 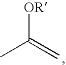
each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “cis” is art-recognized and refers to the arrangement of two atoms or groups around a double bond such that the atoms or groups are on the same side of the double bond. Cis configurations are often labeled as (Z) configurations.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “aliphatic” is art-recognized and refers to a linear, branched, cyclic alkane, alkene, or alkyne. In certain embodiments, aliphatic groups in the present invention are linear or branched and have from 1 to about 20 carbon atoms.

The terms “amine” and “amino” are art-recognized and refer to both unsubstituted and substituted amines, e.g., a moiety that may be represented by the general formulas: 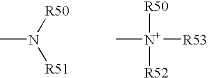
wherein R50, R51 and R52 each independently represent a hydrogen, an alkyl, an alkenyl, —(CH2)m-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH2)m-R61. Thus, the term “alkylamine” includes an amine group, as defined above, having a substituted or unsubstituted alkyl attached thereto, i.e., at least one of R50 and R51 is an alkyl group. The term “amido” is art recognized as an amino-substituted carbonyl and includes a moiety that may be represented by the general formula: 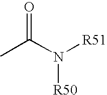
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “carbonyl” is art recognized and includes such moieties as may be represented by the general formulas: 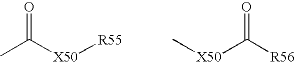
wherein X50 is a bond or represents an oxygen or a sulfur, and R55 and R56 represents a hydrogen, an alkyl, an alkenyl, —(CH2)m-R61 or a pharmaceutically acceptable salt, R56 represents a hydrogen, an alkyl, an alkenyl or —(CH2)m-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂—.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “sulfonamido” is art recognized and includes a moiety that may be represented by the general formula: 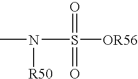
in which R50 and R56 are as defined above.

The term “sulfonyl” is art-recognized and refers to a moiety that may be represented by the general formula: 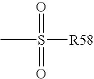
in which R58 is one of the following: hydrogen, alkyl, alkenyl, alkynyl, cycloalkyl, heterocyclyl, aryl or heteroaryl.

The pharmaceutical composition may contain a pharmaceutically-acceptable salt of MNP, analog or derivative. The term “pharmaceutically-acceptable salts” is art-recognized and refers to the relatively non-toxic, inorganic and organic acid addition salts of compounds, including, for example, those contained in compositions of the present invention.

### VI. Combinations

In one aspect of the invention, MNP, or an analog or derivative are administered to a subject in need of improved cognition in combination with a second agent known to be useful for treatment for cognitive impairment. In one embodiment, MNP and the second agent act through different mechanisms and/or affect different aspects of Cognitive Function. The two drugs may exert synergistic effects in a subject when administered in combination.

Suitable drugs for administration in combination with MNP include GABA_{B} receptor antagonists, acetylcholinesterase inhibitors, and NMDA receptor antagonists. Examples are provided below for illustration and not limitation.

Suitable GABA_{B} receptor antagonists include, without limitation, 3-aminopropyl-(n-butyl)-phosphinic acid (“ABPA” or “SGS742”) and others described in U.S. Pat. Nos. 5,300,679 and 5,064,819; 3-{1 (S)-[3-(cyclohexylmethyl) hydroxyphosphinyl)-2(S)-hydroxypropylamino]ethyl} benzoic acid; 3-{1(R)-[3-(cyclohexylmethyl)hydroxyphosphinyl-2(S)-hydroxy-propylamino]ethyl}benzoic acid; phosphinic acid analogues including, without limitation, CGP27492, CGP35024, CGP47656, CGP36216, CGP35348, CGP35913, phaclophen, 2,5 disubstituted-1,4-morpholines; and benzyl-substituted phosphinic acids including, without limitation, CGP54626A, CGP62349, CGP54748A, CGP57076A, CGP67588, CGP80936 (see Enna, 1997, Exp Opin Invest Drugs 6:1319-1325; Froestl et al., 2003, II Farmaco 58:173-83; Bittiger et al., 1993, Trends Pharmacol Sci 14:391-393; Bolser et al., 1995, JPET 274: 1393-1448; Olpe et al., 1990, Eur J. Pharmacol. 187:27-38; U.S. patent publication U.S. 20020091250A1, and PCT patent publication WO 04000326A1).

Suitable acetylcholinesterase inhibitors include, without limitation, donepezil (ARICEPT®), tacrine hydrochloride (COGNEX®), galantamine (REMINYL®), rivastigmine (EXELON®), physostigmine (SYNAPTONO®), metrifonate (PROMEM®), quilostigmine, tolserine, thiatolserine, cymserine, thiacymserine, neostigmine, eseroline, zifrosilone, mestinon, huperzine A and icopezil. See U.S. Pat. No. 4,895,841; U.S. Pat. No. 5,750,542; U.S. Pat. No. 5,574,046; U.S. Pat. No. 5,985,864; U.S. Pat. No. 6,140,321; U.S. Pat. No. 6,245,911; and U.S. Pat. No. 6,372,760.

Suitable NMDA receptor antagonists include, without limitation, memantine hydrochloride (NAMENDA™, Axura®, Ebixa®); D(−)-2-amino-4-phosphonobutyric acid, D(−)-2-amino-7-phosphonoheptanoic acid, D(−)-2-amino-5-phosphonopentanoic acid, DL-2-amino-5-phosphonopentanoic acid, R(−)-3-(2-carboxypiperazin-4-yl)-propyl-1-phosphonic acid, (RS)-3-(2-carboxypiperazin-4-yl)-propyl-1-phosphonic acid, 4-Cl-kynurenine, 7-chloro-kynurenic acid, (−)6-phosphonomethyl-decahydroisoquinoline-3-carboxylic acid, ACPC, aptiganel, besonprodil, BMY-14802, budipine, CGP-37849, CP-101606, conantokin G, CR-3991, CR-2249, CR-3394, delucemine, dexanabinol, dizocilpine, EAA-090, eliprodil, felbamate, fluorofelbamate, FPL-12495, gacyclidine, gavestinel, glycine, harkoseride, HU-211, ibogaine, ipenoxazone, kaitocephalin, ketamine, L-695902, lanicemine, licostinel, ligustizine, midafotel, milnacipran, neboglamine, nebostinel, neramexane, N′-[2-chloro-5-(methylthio)phenyl]-N-methyl-N-[3-(methylthio)phenyl]-guanidine, N′-[2-chloro-5-(methylthio)phenyl]-N-methyl-N-[3-[(R)-methylsulfinyl]phenyl]-guanidine, neramexane, orphenadrine, remacemide, RGH-896, RG-13484, RG-13579, RG-1103, Ro-25-6981, selfotel, seratrodast, spermidine, spermine, topiramate, traxoprodil, UK-240255, ZD-9379, α-amino-2-(2-phosphonoethyl)-cyclohexanepropanoic acid, α-amino-4-(phosphonomethyl)-benzeneacetic acid, N(1)-(benzyl)cinnamamidine, and 4-benzyl-4-hydroxy-N-(hydroxyphenoxyalkyl)-piperidine. Certain of these NMDA receptor antagonists are described in U.S. patent publications 20040082543 and 20040058896, and in the Investigational Drug Database (www.iddb.com).

Drugs are administered to a subject “in combination” when the drugs are administered as part of the same course of therapy. In this context, a “course of therapy” refers to administration of combinations of drugs believed by the medical professional to work together additively, complementarily, synergistically, or otherwise to produce a more favorable outcome than that anticipated for administration of a single drug. A course of therapy can be for one or a few days, but more often extends for several weeks.

The term “simultaneous administration,” or “co-administration” and equivalents as used herein, means that the MNP, analog or derivative and the second agent are administered, for example as a co-formulation or as separate compositions, with a time separation of no more than about 15 minutes, such as no more than about 10 minutes. When the drugs are administered simultaneously, the MNP, analog or derivative and the second agent may be contained in the same dosage (e.g., a unit dosage form comprising both the MNP, analog or derivative and second agent) or in discrete dosages (e.g., the MNP, analog or derivative is contained in one dosage form and the second agent is contained in another dosage form).

In a related aspect, the invention provides a pharmaceutical composition, e.g., in unit dosage form, comprising MNP, or an analog or derivative thereof, and a second compound.

The invention having been generally described, may be more readily understood by reference to the following examples, which are included merely for purposes of illustration of certain aspects and embodiments of the present invention, and are not intended to limit the invention in any way.

### VII. Examples

A. Introduction

Advanced age is a major risk factor for a variety of conditions with cognitive impairment (e.g., Alzheimer's Disease, Mild Cognitive Impairment [MCI] and Age Related Cognitive Decline [ARCD]). Animal models serve as an important resource for developing treatments for age-related cognitive impairments, since features that characterize cognitive impairments in animal models likely extend to cognitive impairments in humans. Of available models, a Long-Evans rat model of cognitive impairment is particularly well suited for distinguishing the difference between illness and normal aging: Extensive behavioral characterization has identified a naturally occurring form of cognitive impairment in an outbred strain of aged Long-Evans rats (Charles River Laboratories; Gallagher et al., 1993, Behav. Neurosci. 107:618-26). In a behavioral assessment with the Morris Water Maze (MWM), rats learn and remember the location of an escape platform guided by a configuration of spatial cues surrounding the maze. The cognitive basis of performance is tested in probe trials using measures of the animal's spatial bias in searching the location of the escape platform. Aged rats in the study population have no difficulty swimming to a visible platform, but an age-dependent impairment is detected when the platform is camouflaged, requiring the use of spatial information. Performance for individual aged rats in the outbred Long-Evans strain varies greatly, with a proportion of those rats performing on a par with young adults but approximately 40-50% falling outside the range of young performance. This variability among aged rats reflects reliable individual differences. Thus, within the aged population some animals are cognitively impaired and designated aged impaired (AI) and other animals are not impaired and are designated aged unimpaired (AU). See, e.g., Colombo et al., 1997, Proc. Natl. Acad. Sci. 94: 14195-99; Gallagher and Burwell, 1989, Neurobiol. Aging 10: 691-708; Rapp and Gallagher, 1996, Proc. Natl. Acad. Sci. 93: 9926-30; Nicolle et al., 1996, Neuroscience 74: 741-56; and Nicolle et al., 1999, J. Neurosci. 19: 9604-10.

Using this animal model to identify genes implicated in age-related changes in cognitive function, it was determined that expression of genes encoding glutamate transporter proteins, GLT1 and GLAST [human homologs, EAAT-2 and EAAT-1, respectively] is significantly increased in aged individuals with unimpaired cognitive function relative to young individuals and aged individuals with impaired cognitive function. See Gallagher et al., U.S. patent application Ser. No. 10/722,357 (filed Nov. 24, 2003 and published as U.S. Patent Publication 20040191803 on Sep. 30, 2004) which is incorporated by reference in its entirety. It was also demonstrated that administration of ceftriaxone, an agent that increased expression of GLT1 in young rats, resulted in improvements of cognitive function of aged rats. We show here that administration of (R)-(−)-5-methyl-1-nicotinoyl-2-pyrazoline (MNP; also called “MS-153”) increased GLT1 protein expression in the hippocampus of young rats. When administered to aged-impaired (AI) rats, MNP reduced the severity of age-related cognitive impairment and improved cognitive function.

### B. Example 1

### MNP Enhances the Cognitive Performance of Aged Rats

Effect of MNP Treatment on GLT1 Protein Expression in Young Rats

Administration of MNP: Twelve young Long-Evans rats, weighing approximately 400-500 grams, received treatment with either vehicle (0.9% saline; n=6) or MNP (50 mg/kg/day; n=6). The rats were administered MNP and vehicle continuously for one week via an osmotic minipump (Alzet, model 2ML1) implanted subcutaneously on the back, slightly posterior to the scapulae. After 7 days of treatment, the rats were sacrificed, their brains were removed, the hippocampi dissected out, frozen on dry ice and sent for Western blot analysis. The implanted minipump was also removed to verify proper drug delivery by measuring residual volume.

Preparation of brain tissue; Hippocampi were homogenized in 2 ml sucrose buffer (20 mM Tris pH 7.4, 10% sucrose, complete protease inhibitor cocktail mini-tablets [Roche cat#1-836-153]) for 25 seconds (Omni 115V Tissue Homogenizer TH-115). SDS (at 2%) was immediately added to the homogenate, and the samples were sonicated (Branson Sonifier 250; 10 pulses each). Extracts were total cellular extracts solubilized in SDS and include both cytoplasmic and membrane-bound GLT1.

Western blot analysis: Immunoblotting was performed using a LICOR Odyssey based system which uses IRDye 800 and Alexa Fluor 680 labeled secondary antibodies and signal is detected by diode lasers sensitive to infrared emissions of different wavelength. This system has been established to provide highly sensitive protein detection with linear changes in signal intensity over several orders of magnitude protein dose, providing superior quantitative capabilities compared with chemiluminescence based methods. Samples were electrophoresed on a 10% SDS-polyacrylamide gel, blotted electrophoretically to Immobilon-P, and blocked in 0.1% casein, 0.2×PBS (no Tween). Western blots were blocked in 0.1% casein, 0.2×PBS (no Tween). Blots were probed with a Calbiochem antibody (cat# PC154) raised to the COOH terminus of GLT1 (0.4-1 μg of protein) or an antibody to the specific splice variant, GLT1B. Scanning and densitometry was then done with the LICOR Odyssey software system.

MNP Increases GLT1 Protein Expression in the Rat Hippocampus

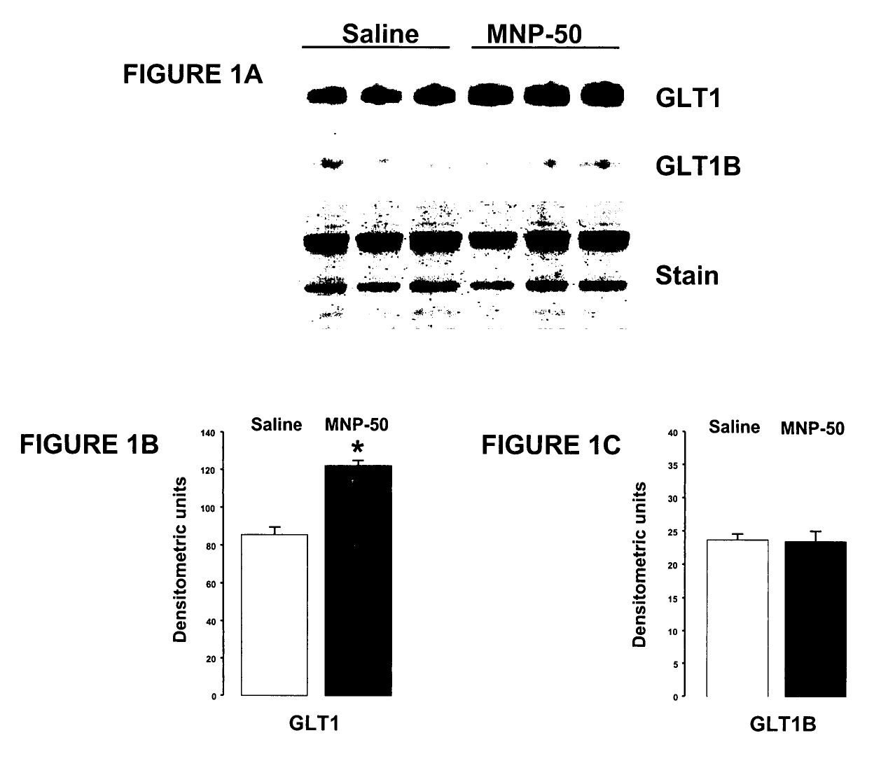

FIG. 1 shows representative Western blots of hippocampal tissue from rats treated with saline and those treated with 50 mg/kg/day MNP for 7 days. GLT1 immunoreactivity was significantly higher in the MNP-treated animals compared to vehicle controls. No difference was observed between these groups in the level of GLT1B immunoreactivity. Coomassie blue staining shows equal protein loading across all samples. Pooled data (n=5 for each condition) are summarized in the histograms illustrating the significant increase in GLT1 protein expression induced by 7 days of treatment with 50 mg/kg MNP.

Behavioral Characterization of Young, Aged-Impaired (AI) and Aged-Unimpaired (AU) Rats in Morris Water Maze (MWM) and Radial Arm Maze (RAM)

Behavioral tests were performed on young (4-6 months old) and aged (25-27 months old) pathogen-free male Long-Evans rats. Aged rats were tested in the MWM, followed by training and testing in the radial arm maze (RAM) to assess test-retest reliability for individual differences in cognitive function across the two tasks.

The MWM apparatus consists of a large, circular pool (diameter 1.83 m; height, 0.58 m) filled with water (27° C.) that has been made opaque through the addition of non-toxic pigment or some other substance. In the typical “hidden platform” version of the task, rats are trained to find a camouflaged white escape platform (height, 34.5 cm) that is positioned in the center of one quadrant of the maze just 1.0 cm below the water surface. This platform could be retracted to the bottom of the tank or raised to its normal position from outside the maze during behavioral testing. The location of this platform remained constant from trial to trial. Because there were no local cues that marked the position of the platform, the rat's ability to locate it efficiently from any starting position at the perimeter of the pool depended on using information surrounding the maze. The maze was surrounded by black curtains with white patterns affixed to provide a configuration of spatial cues. A second platform (height 37.5 cm), with its surface painted black was elevated 2 cm above the water surface during cue training, the version of the task used to control for factors unrelated to cognition. The behavior of a rat in the pool was recorded by a camera suspended 2.5 m above the center of the pool, connected to a video tracking system (HVS Image Advanced Tracker VP200) and a PC computer running HVS software developed by Richard Baker of HVS Image, Hampton, UK.

The MWM protocol was optimized for sensitivity to the effects of aging on cognition and for measures of reliable individual differences within the aged population of out-bred Long-Evans rats (Gallagher M, Burwell R, Burchinal M. Behav. Neurosci. 107:618-626; 1993).

Rats received three trials per day for 8 consecutive days, using a 60 sec intertrial interval. On each training trial, the rat was released in the maze from one of four equally spaced starting positions around the perimeter of the pool. The starting position varied from trial to trial, thus preventing the use of a response strategy (e.g. always turning left from the start location to locate the escape platform). If a rat did not locate the escape platform within 90 sec on any trial, the experimenter guided the rat to the platform, where it remained for 30 sec. Every sixth trial consisted of a probe trial to assess the development of spatial bias in the maze. During these trials, the rat swam with the platform retracted to the bottom of the pool for 30 sec, at which time the platform was raised to its normal position for completion of an escape trial. At the completion of the protocol using the hidden platform, rats were assessed for cue learning using the visible platform. The location of this platform varied from trial to trial in a single session of 6 training trials.

The proximity of the animal's position with respect to the goal was used for analysis of training trial and probe trial performance. The proximity measure was obtained by sampling the position of the animal in the maze (10×/sec) to provide a record of distance from the escape platform in 1 sec averages. For both probe trials and training trials, a correction procedure was implemented so that trial performance was relatively unbiased by differences in distance to the goal from the various start locations at the perimeter of the pool. In making this correction the average swimming speed was calculated for each trial (path length/latency). Then the amount of time required to swim to the goal at that speed from the start location used on the trial was removed from the record prior to computing trial performance, i.e. cumulative distance on training trials and average distance from the goal on probe trials. Thus, scores obtained using the proximity measure are designed to reflect search error, representing deviations from an optimal search, i.e. direct path to the goal and search in the immediate vicinity of that location during probe trials.

Computer records of video-tracking were compiled to provide data on each rat's performance in the maze. Measures on training trials and probe trials were analyzed by Analysis of Variance.

The performance during training with the hidden, camouflaged platform differed between the groups of young and aged rats [F(1,23)=12.69, p<0.002]. No difference between the groups occurred for the cue training trials with a visible platform. Latencies to escape during cue training averaged 9.36 seconds for young and 10.60 seconds for the aged rats.

The average proximity measure on interpolated probe trials was used to calculate a spatial learning index for each individual subject as described in detail in Gallagher et al., 1993, Behav. Neurosci. 107:618-26. When a rat rapidly learned to search for the platform close to its position, it's spatial learning index is low. Overall, aged rats differed from young [F(1,23)=15.18, p<0.001]. Aged rats were classified as either unimpaired or impaired relative to the learning index profile of the young study population. Aged rats that fall within the normative range of young rats (index scores <241) were designated aged-unimpaired. The remaining aged subjects that have index scores outside the range of young performance were designated aged-impaired.

To evaluate test-retest reliability, animals characterized in the MWM were tested in the RAM: Each arm (7×75 cm) of the elevated eight arm radial maze projected from each facet of an octagonal center platform (30 cm diameter, 51.5 cm height). Clear side walls on the arms were 10 cm high and were angled at 65° to form a trough. A food well (4 cm diameter, 2 cm deep) was located at the distal end of each arm. Blocks constructed of Plexiglas (30 cm H×12 cm W) could be positioned to block entry to any arm. Numerous extra maze cues were provided in the room surrounding the apparatus and lighting was provided by overhead fixtures.

Rats were first habituated to the maze for an 8 min session on four consecutive days. In each of these sessions food rewards were scattered on the RAM, initially on the center platform and arms and then progressively confined to the arms. After this habituation phase, a standard training protocol was used in which a food pellet was located at the end of each arm. Rats received one trial each day for 18 days; each daily trial terminated when all eight food pellets had been obtained or when either 16 choices were made or 15 min had elapsed. An error consisted of returning to an arm (all four paws on the arm) from which food had already been obtained. After completion of this phase, the memory demand of the task was increased by imposing a delay during the trial. At the beginning of each trial three arms were blocked. The identity and configuration of the blocked arms was varied across trials. Rats were allowed to obtain food on the five arms to which access was permitted at the beginning of the trial. The rat was then removed from the maze for 60 s, during which time the barriers on the maze were removed, thus allowing access to all eight arms. Rats were then placed back onto the center platform and allowed to obtain the remaining food rewards.

A memory error occurred during test trials using a 60 second delay when a rat returned to one of the five arms that was already visited prior to the delay. Each rat's performance was averaged across four consecutive test trials. Parametric statistics (unpaired t-tests) were used to compare performance between young and aged groups. Correlational analysis (Pearson's r) was used to examine the relationship between performance of aged rats (N=10) in the Morris water maze (learning index scores) and radial-arm maze (memory errors).

The performance of young adult rats in the delay version of the RAM varies as a function of the delay interval, ranging from 60 seconds to eight hours (Chappell et al. Neuropharmacology 37: 481-488, 1998). Aged rats previously characterized in the MWM, committed more memory errors after a 60 second delay relative to young rats (p<0.025). On average young rats committed 0.17 errors, whereas aged rats committed an average of 1.52 errors. The ten aged rats, however, exhibited a wide range of performance on the RAM. A significant relationship was found between the initial MWM characterization and memory performance in the RAM (r value=0.82).

Effect of MNP Treatment on the Performance of Aged-Impaired Rats in the Spatial Memory Version of the Morris Water Maze

Spatial memory version of the Morris water maze: Behavioral testing is conducted by an experimenter who is blind to drug treatment. Fourteen 25-27 month-old Long-Evans rats, previously characterized as cognitively impaired (AI rats), were tested in a modified version of the Morris water maze (MWM) task using the same MWM apparatus as described above. Unlike the traditional protocol wherein the platform location remained constant throughout training, the escape platform location in this spatial memory version of the task varied from day-to-day (in one of nine different positions).

AI rats were given six training trials per day with a 60-sec intertrial interval. On each training trial, the rat was released in the maze from one of four equally spaced starting positions around the perimeter of the pool. If the rat did not locate the escape platform within 90 sec on any trial, the experimenter guided the rat to the platform, where it remained for 30 sec. Following the six training trials, the rat is returned to its home cage and placed in the animal housing room. After a delay of four hours, the rat is given one additional testing trial (the “retention trial”) with the escape platform located in the same position as in the 6 training trials. The length of the rat's swim path to reach the escape platform is measured in all six training trials and the retention trial. Spatial memory in this task is measured by comparing the swim path lengths in the retention trial with the sixth (and final) training trial. If the swim path for the retention trial is significantly longer than in the final training trial, the rat has forgotten the location of the platform. A greater difference in performance in these two trials represents a greater degree of forgetting. After two days of acclimation to these procedures, this test protocol is given for nine consecutive days, with the position of the escape platform moved daily.

MNP enhances the cognitive performance of aged rats in the spatial memory version of the MWM: Following characterization for cognitive status with a traditional MWM protocol, impaired aged rats were assigned to one of two treatment conditions (vehicle controls or MNP). Mean learning index scores in these two groups did not differ from each other (AI(vehicle group): 274.7±7.0, AI(MNP group): 269.4±7.9). AI rats were pretreated with either vehicle or MNP for 9 days prior to behavioral testing. Osmotic minipumps (Alzet) were filled with either vehicle (0.9% saline) or MNP (50 mg/kg/day) and implanted subcutaneously. Initially, Alzet pumps that administer compound for one week were implanted; after seven days, these pumps (now empty) were removed and replaced with full two-week pumps allowing delivery of either MNP or saline for a total of 21 days. On Day 9 of treatment with either vehicle or MNP, MWM testing was begun. Data were collected in two replications with vehicle- and drug-treated rats represented in each. A total of 9 vehicle-treated and 5 MNP-treated rats completed the entire protocol. Several rats in both treatment groups, however, died during the execution of the study. This attrition is attributable to the age of the test animals (25-26 mo), the procedures used for drug delivery (two minipump implantation surgeries requiring anesthesia), and possibly the extended testing under moderately stressful conditions of swim escape in a water maze. There was no indication that attrition was related to MNP exposure.

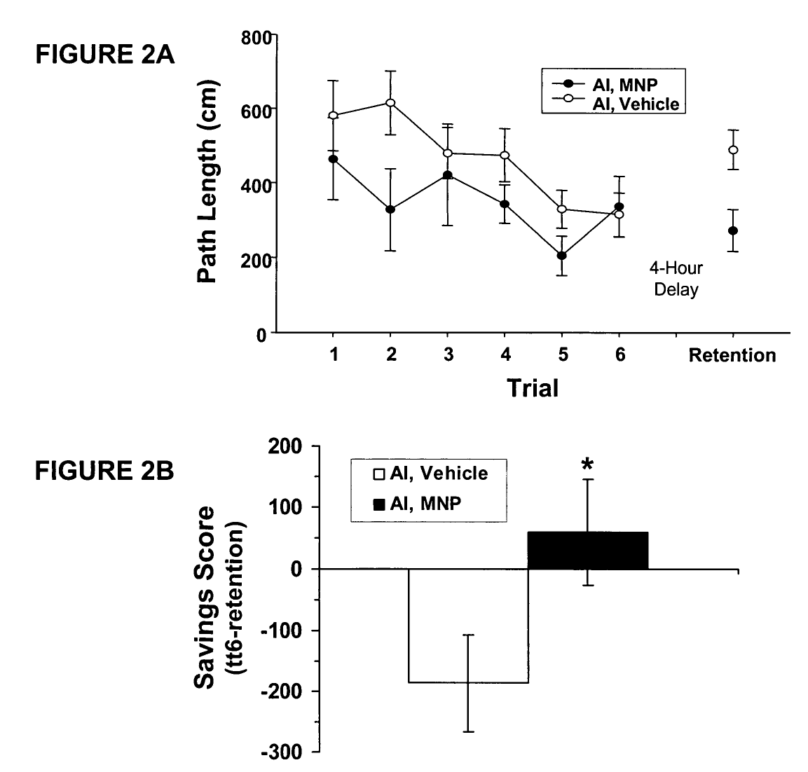

Aged impaired rats that received MNP (at 50 mg/kg/day) performed significantly better in retention trials than did vehicle-treated controls (FIG. 2A-B). Animals in both groups improved performance within the day's training session and attained equal performance by the last training trial (FIG. 2A). A two-way analysis of variance indicated no significant difference between the groups on training trials 1-6. There was a significant overall effect of trials [F(5,12)=2.84, p<0.05], reflecting the improvement with training. However, the performance of vehicle-treated rats deteriorated significantly during the 4-hour delay between the final training trial and the retention trial. In comparison, there was no significant difference in performance on the final training trial and the retention trial in rats treated with MNP (50 mg/kg/day), suggesting that MNP enhanced spatial memory retention in this task. Analysis of performance on the retention trial indicated a significant difference between the groups, such that the MNP-treated rats maintained a proficient ability to locate the escape platform, whereas the vehicle-treated rats did not [t(12)=2.63, p<0.05]. To further quantify spatial memory in this task on a within-subject basis, “savings scores” were calculated for each rat by subtracting the swim path length observed in the retention trial from the path length observed in the final training trial (FIG. 2B). A savings score at or near zero (0) indicates that there was no decline in performance between the two trials, suggesting intact spatial memory retention. Poor memory retention was represented by savings scores substantially less than zero (0). Aged impaired rats treated with 50 mg/kg/day MNP retained memory for the platform location significantly better than vehicle-treated controls (Mann Whitney U test, one tailed; U=10, p<0.05). The data presented in FIG. 2 are averaged across three different experimental blocks (i.e. three different escape platform locations, etc.) conducted on Days 9-11 of treatment with either MNP or vehicle. In a separate experiment, young rats displayed a similar level of spatial memory savings in retention trials. Taken together, these data suggest that treatment with MNP can return the performance of AI rats to the level of young rats in this version of the Morris water maze.

### Example 2

### MNP is Highly Bioavailable when Administered Orally

Pharmacokinetics of MNP in the Rat

Pharmacokinetic experiments were conducted. MNP was dissolved in saline solution (0.9% NaCl) and administered to male Sprague-Dawley rats (weighing 250-315 grams) with vascular catheters surgically placed in both jugular veins (Charles River, Wilmington, Mass.). The catheter on the left jugular vein was used for intravenous infusion of 5 mg/kg MNP (in the animals that received the i.v. dosing), while the catheter on the right jugular vein was used for blood sample collection. Oral doses of MNP (50 and 200 mg/kg) were administered by oral gavage. Blood samples were taken at ten different time points: immediately prior to dosing (control), 5, 15, 30, 60, 120, 240, 480, 720, and 1440 minutes post-drug administration. The samples were collected in heparin-coated microtainers, spun down in a microcentrifuge (14,000 rpm for 7 minutes) to separate out the blood plasma and frozen until analyzed. Plasma levels were quantitated by LC/MS/MS analysis (Applied Biosystems/MDS SCIEX API 3000). The plasma kinetics of intravenous and oral doses of MNP are described in Table I. For each dose of MNP, Table I shows the mean plasma levels (±s.e.m.) detected in three rats. Orally administered MNP was rapidly absorbed, as evidenced by the presence of significant levels of the compound in the blood plasma at the 5-min time point. For the 50 mg/kg oral dose, a secondary peak was evident at about 8 hours suggesting a complex absorption/elimination pattern. The 200 mg/kg oral dose also displayed complex pharmacokinetics with significant plasma levels of MNP present at 24 hours post-administration.

The pharmacokinetic parameters Cₘₐₓ, tₘₐₓ, area-under-the-curve (AUC), clearance, and half-life (t_{1/2}) were calculated for oral doses of MNP (50 and 200 mg/kg) and are described in Table II. These parameters are compared to the intravenous dose (5 mg/kg) to determine the oral bioavailability (Fₒᵣₐₗ) of MNP in the rat. With no samples collected before 5 minutes post-administration, these data were not sufficient for precise calculation of pharmacokinetic parameters, so only approximate values are presented. The roughly 30-minute terminal half-life calculated for the intravenous dose of MNP (5 mg/kg) was similar to previously reported data (Umemura et al., Stroke, 1996; 27:1624-1628). Based on the calculations of the AUC and Fₒᵣₐₗ for both oral doses analyzed, MNP is highly bioavailable when administered orally.

TABLE I

| | MNP plasma levels (ng/ml) | | |
|---|---|---|---|
| Time (min) | 5 mg/kg i.v. | 50 mg/kg p.o. | 200 mg/kg p.o. |
| 5 | 6550 ± 32 | 15967 ± 1719 | 50267 ± 2642 |
| 15 | 4530 ± 60 | 16033 ± 933 | 41300 ± 3208 |
| 30 | 2877 ± 95 | 13633 ± 1117 | 36467 ± 5573 |
| 60 | 1300 ± 75 | 9977 ± 976 | 21633 ± 3790 |
| 120 | 272 ± 9 | 6140 ± 1985 | 11000 ± 2187 |
| 180 | 51 ± 3 | 4180 ± 1639 | 5697 ± 1968 |
| 240 | 13 ± 1 | 2470 ± 792 | 5837 ± 1436 |
| 480 | 2 ± 1 | 2853 ± 755 | 2920 ± 343 |
| 720 | 3 ± 2 | 536 ± 327 | 1156 ± 360 |
| 1440 | 2 ± 0.5 | 9 ± 6 | 6250 ± 2829 |

TABLE II

| MNP | | | | | | |
|---|---|---|---|---|---|---|
| Dose and route of | ˜Cₘₐₓ | ˜Tₘₐₓ | ˜AUC | ˜Clearance | ˜t_{1/2} | ˜Bioavailability^{†} |
| administration | (ng/mL) | (min) | (ng/mL*min) | (mL*min⁻¹) | (min⁻¹) | (Fₒᵣₐₗ; %) |
| 5 mg/kg i.v. | 6550 | 5 | 2.53e+05 | 5.6 | 30 | — |
| 50 mg/kg p.o. | 16033 | 10 | 3.01e+06 | 4.8 | 150 | 119 |
| 200 mg/kg p.o. | 50267 | 5 | 8.07e+06 | 7.8 | 90 | 80 |

^{†}Effects of saturation and accumulation are unknown.
Equations: Clearance = Dose/AUC; Fₒᵣₐₗ = (AUC oral/AUC iv_{avg})* (dose iv/Dose oral)

While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

The contents of each of the references cited in the present application, including publications, patents, and patent applications, are herein incorporated by reference in their entirety.

What is claimed is:

1. A method for improving cognitive function in a subject in need of such improvement, comprising administering a therapeutically effective amount of (R)-(−)-5-methyl-1-nicotinoyl-2-pyrazoline (MNP) or an analog thereof to the subject.

2. The method of claim 1 wherein the subject exhibits age-related cognitive decline.

3. The method of claim 2 wherein the subject is a human diagnosed as having Alzheimer's Disease (AD), Mild Cognitive Impairment (MCI) or Age Related Cognitive Decline (ARCD).

4. The method of claim 2 wherein (R)-(−)-5-methyl-1-nicotinoyl-2-pyrazoline (MNP) is administered.

5. The method of claim 2 wherein an analog of (R)-(−)-5-methyl-1-nicotinoyl-2-pyrazoline is administered, said analog having the formula: 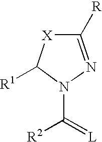
I
wherein, independently for each occurrence:
R is H, C₁-C₁₀ alkyl, C₂-C₁₀ alkenyl, C₂-C₁₀ alkynyl, aryl, or aralkyl;
R¹ is H, C₁-C₁₀ alkyl, C₂-C₁₀ alkenyl, C₂-C₁₀ alkynyl, aryl, or aralkyl;
R² is a heterocyclic or heteroaryl ring comprising from 1-4 heteroatoms selected from the following: N, O, or S;
L is O, S, or NR; and
X is CR₂, O, or S.

6. The method of claim 2 wherein the subject is not diagnosed with stroke.

7. The method of claim 2 wherein a dose of between 0.1 μg and 10 grams is administered.

8. The method of claim 2 wherein MNP is administered orally at a daily dosage of from 100 mg/day to 700 mg/day.

9. The method of claim 8 wherein MNP is administered for at least two months.

10. The method of claim 2 wherein MNP is administered in combination with another agent known to be useful for treatment for cognitive impairment.

11. The method of claim 10 wherein the other agent is selected from the group consisting of a GABA_{B} receptor antagonist, an acetylcholinesterase inhibitor, and an NMDA receptor antagonist.

12. The method of claim 11 wherein the MNP and the other agent are administered at the same time or as a co-formulation.
